(19) United States
(12) Patent Application Publication
(10) Pub. No.: US 2025/0099642 A1
(43) Pub. Date: Mar. 27, 2025

(54) APPARATUS FOR DISPENSING LIQUID SOAP AND AIR FRAGRANCE

(71) Applicant: Roy Peer, Riverview, FL (US)

(72) Inventor: Roy Peer, Riverview, FL (US)

(21) Appl. No.: 18/624,897

(22) Filed: Apr. 2, 2024

Related U.S. Application Data
(60) Provisional application No. 63/585,925, filed on Sep. 27, 2023.

(51) Int. Cl. A61L 9/12; A47K 5/12

(52) U.S. Cl. CPC A61L 9/12; A47K 5/1205; A61L 2209/13

(57) ABSTRACT

An apparatus includes a liquid dispensing unit and a fragrance dispensing unit. The liquid dispensing unit can dispense liquid, such as soap. A pump can be included for dispensing the soap. The fragrance dispensing unit is coupled to the bottom of the liquid dispensing unit. The fragrance dispensing unit can release fragrance and include suitable mechanisms for the release of fragrance.

### CROSS-REFERENCE TO RELATED APPLICATIONS

This application claims priority from a U.S. Provisional Patent Appl. Ser. No. 63/585,925, filed on Sep. 27, 2023, which is incorporated herein by reference in its entirety.

### FIELD OF INVENTION

The present invention relates to an apparatus for dispensing liquid and air freshener, and more particularly, the present invention relates to an apparatus having a refillable liquid dispenser and an integral air fragrance dispenser.

### BACKGROUND

Liquid dispensers are widely used for a variety of purposes. A widespread use of liquid dispensers is for dispensing liquid soap. Also, referred to as soap dispensers, they are an essential utility item in restrooms. Soap dispensers are commonly used in both residential and public washrooms.

Also, it is common to use some kind of air fresher in the restrooms to mask bad odor. A variety of air fresheners are available in the market, such as gel-based air fresheners which slowly release air fragrance for days. Electronic fragrance dispensers are also known to disperse air fresheners in the air for short durations.

Although both i.e., soap dispensers and fragrance dispensers are commonly used, purchasing them separately is costly. Moreover, there are space restraints in placing an air fresher in a restroom.

A need is therefore appreciated for an apparatus that can dispense both the soap and fragrance in the air.

### SUMMARY OF THE INVENTION

The following presents a simplified summary of one or more embodiments of the present invention to provide a basic understanding of such embodiments. This summary is not an extensive overview of all contemplated embodiments and is intended to neither identify key or critical elements of all embodiments nor delineate the scope of any or all embodiments. Its sole purpose is to present some concepts of one or more embodiments in a simplified form as a prelude to the more detailed description that is presented later.

The principal object of the present invention is therefore directed to an apparatus for dispensing liquid and fragrance.

Another object of the present invention is that the apparatus is compact.

Still another object of the present invention is that the apparatus has an aesthetic appearance.

Yet another object of the present invention is that the liquid for dispensing can be refilled.

A further object of the present invention is that the fragrance unit can be refilled or replaced.

Still, a further object of the present invention is that the apparatus saves space in a washroom.

Yet a further object of the present invention is that the amount of fragrance released by the fragrance unit can be controlled.

In one aspect, disclosed is an apparatus comprising a liquid dispensing unit and a fragrance dispensing unit. The liquid dispensing unit comprises a container; and a pump for dispensing the liquid contained in the container. The fragrance dispensing unit is coupled to the bottom of the liquid dispensing unit. The fragrance dispensing unit comprises a receptacle configured to contain fragrance substances; a lid covering an opening in the receptacle; and one or more holes in a wall of the receptacle or the lid, wherein one or more holes are for releasing the fragrance. The liquid is soap.

In one aspect, one or more holes comprise multiple holes in the lid of the fragrance dispensing unit. The fragrance dispensing unit is configured to rotate from a closed state to up to 90 degrees, wherein the lid of the fragrance dispensing unit is covered by the bottom of the container in the closed state. Wherein a degree of rotation of the fragrance dispensing unit relative to the liquid dispensing unit from the closed state up to 90 degrees is proportional to several holes exposed for releasing the fragrance, wherein the fragrance release increases with rotation of the fragrance dispensing unit.

In one aspect, each of the container and the receptacle has one or more transparent or translucent windows to check the level of the liquid inside the container and the level of the fragrance substance inside the receptacle. The multiple holes in the lid are sealed in the closed state by the bottom of the container preventing any leakage of the fragrance substance.

In one aspect, disclosed is a method for dispensing a liquid and fragrance, the method comprising providing the apparatus as described above. The method further comprises dispensing the liquid from the liquid dispensing unit; and dispensing, continuously or periodically, fragrance by diffusion in air, from the fragrance dispensing unit.

### BRIEF DESCRIPTION OF DRAWINGS

The accompanying figures, which are incorporated herein, form part of the specification and illustrate embodiments of the present invention. Together with the description, the figures further explain the principles of the present invention and to enable a person skilled in the relevant arts to make and use the invention.

FIG. 1 is a perspective view of an apparatus, according to an exemplary embodiment of the present invention.

FIG. 2 shows the apparatus in which the air fragrance unit is rotated for releasing the fragrance, according to an exemplary embodiment of the present invention.

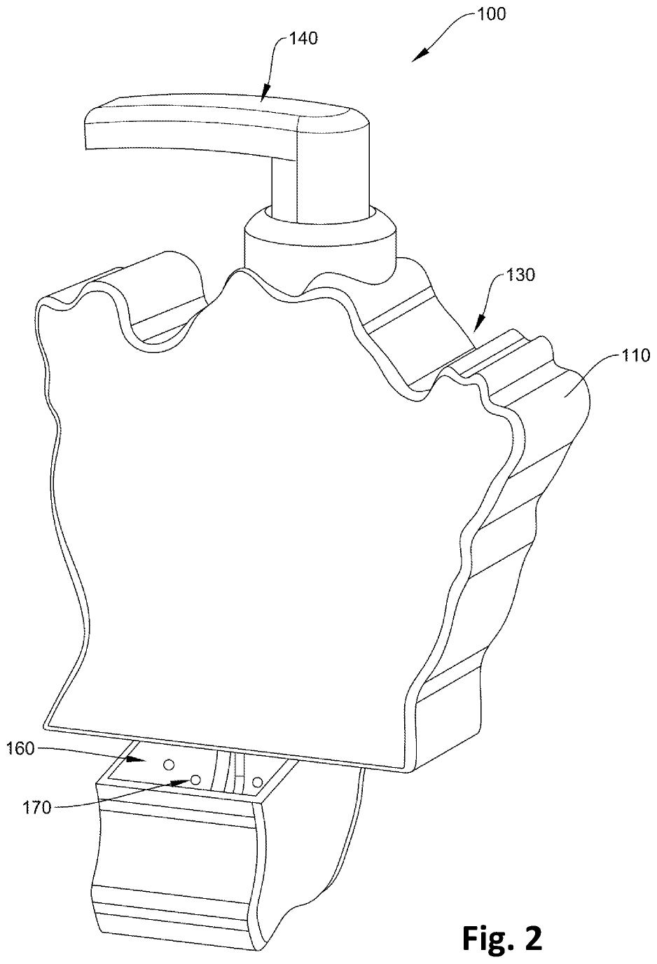

FIG. 3 shows another implementation of the apparatus, according to the present invention.

FIG. 4 shows an exploded view of the apparatus, according to an exemplary embodiment of the present invention.

FIG. 5 shows a bottom and side perspective view of the liquid dispensing unit of the apparatus, according to an exemplary embodiment of the present invention.

FIG. 6 shows a perspective view of the fragrance dispensing unit of the apparatus, according to an exemplary embodiment of the present invention.

### DETAILED DESCRIPTION

Subject matter will now be described more fully hereinafter with reference to the accompanying drawings, which form a part hereof, and which show, by way of illustration, specific exemplary embodiments. Subject matter may, however, be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, the subject matter may be embodied as methods, devices, components, or systems. The following detailed description is, therefore, not intended to be taken in a limiting sense.

The word “exemplary” is used herein to mean “serving as an example, instance, or illustration.” Any embodiment described herein as “exemplary” is not necessarily to be construed as preferred or advantageous over other embodiments. Likewise, the term “embodiments of the present invention” does not require that all embodiments of the invention include the discussed feature, advantage, or mode of operation.

The terminology used herein is to describe particular embodiments only and is not intended to be limiting to embodiments of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

The following detailed description includes the best currently contemplated mode or modes of carrying out exemplary embodiments of the invention. The description is not to be taken in a limiting sense but is made merely to illustrate the general principles of the invention since the scope of the invention will be best defined by the allowed claims of any resulting patent.

Disclosed is an apparatus for dispensing liquid and fragrance, and a method of use thereof. The disclosed apparatus includes a liquid dispensing unit and a fragrance dispensing unit. The fragrance dispensing unit can be coupled to the bottom of the liquid dispensing unit.

The liquid dispensing unit can include a container for containing the liquid. The liquid can be soap; however, any type of liquid is within the scope of the present invention. The container can be made from any suitable material, such as plastic and metal. The shape of the container can be varied for aesthetic purposes. For example, the container can be cylindrical. However, the base of the container can be flat. For aesthetic appearances, suitable design and coating can be applied to the outer surface of the container.

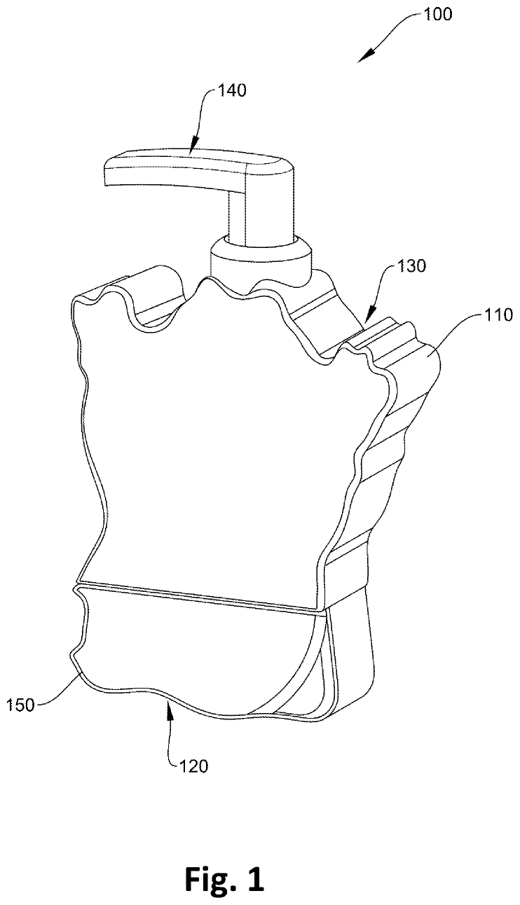

The liquid dispensing unit may also include a pump mechanism for dispensing the liquid, such as soap from the container. The pump mechanism can be manual or electronic, both mechanisms are known in art, and any such pump mechanism can be used without departing from the scope of the present invention. The pump mechanism can be coupled to the top of the container. Referring to FIG. 1 shows apparatus 100 having a liquid dispensing unit 110, and a fragrance dispensing unit 120. The liquid dispensing unit 110 includes a container 130 and a pump member 140.

The fragrance dispensing unit can be coupled to the bottom of the container of the liquid dispensing unit. The fragrance dispensing unit can contain the fragrance-releasing substance that may release the fragrance slowly over a long period. Such fragrance-releasing substances are known in the art and any such fragrance-releasing substance can be used without departing from the scope of the present invention. Moreover, fragrance of any type of aroma can be used. In certain implementations, the fragrance dispensing unit may include a receptacle 150 which may contain the fragrance substance. The receptacle may resemble the shape of the container for aesthetic appearance; however, any shape of the receptacle is within the scope of the present invention. It is to be noted that the receptacle may be of a short height and less volume than the container, however, the shape and volume of the receptacle can be varied without departing from the scope of the present invention. Moreover, designs and coatings can also be applied to the outer surface of the receptacle like that over the container. The base of the receptacle may be flat which allows placing the apparatus on a flat surface.

The receptacle can have a lid 160 to cover the open top of the receptacle or any opening in the receptacle. The lid can have holes 170 for the fragrance to be released into the atmosphere. The number of holes can be varied, wherein the number of holes is proportional to the amount of fragrance released into the air. The number of holes exposed for releasing the fragrance in the air at a given time can be controlled. For the desired level of fragrance, the holes can be strategically placed to allow the diffusion of the fragrance when exposed. These holes are covered or exposed based on the twist mechanism and position of the liquid dispensing unit.

In certain implementations, the fragrance dispensing unit can be refilled with a new fragrance substance, when the fragrance substance in the receptacle gets exhausted. For example, the receptacle can be removed from the lid and refilled with a new fragrance substance. The receptacle can have a sticker for covering the holes and the sticker can be removed before installing the fragrance dispensing unit.

In certain implementations, the fragrance dispensing unit can be replaceable. For example, the exhausted fragrance dispensing unit can be replaced by the new fragrance dispensing unit. In either case, the fragrance dispensing unit can be sealed to prevent any leakage of the fragrance. The fragrance substance can be gel, solid block, or liquid.

The fragrance dispensing unit can be rotatably coupled to the bottom of the container. A twist fastener can be used to couple the fragrance dispensing unit to the liquid dispensing unit. The twist fastener allows rotating the fragrance dispensing unit relative to the liquid dispensing unit. The rotation of the fragrance dispensing unit reveals more holes for the diffusion of the fragrance substances. This twist fastener can be equipped with soft tactile clicks or stops, that give the users a sense of preset intensity levels. Rather than the free rotation, the clicks while rotation allows the user to have better control.

Depending on the user's preference for the intensity of aroma, a minimal twist reveals fewer holes for a subtle fragrance, while a maximum twist uncovers all vents for a more potent aroma. An alignment marker or scale can be etched or printed on the surface to indicate the level of intensity visually.

The apparatus can further include a seal to prevent unwanted fragrance release during non-use. For example, a rubber or silicone seal can be incorporated into the fragrance dispensing unit that may effectively block the holes in the fully closed position.

In certain implementations, the fragrance dispensing unit can also include a slider or lever incorporated on one side of the receptacle. As the user slides the slider up or down, it reveals or conceals the holes in the receptacle for the fragrance to diffuse.

In certain implementations, the tactile feedback allows for easy adjustments without needing to handle or turn the entire bottle.

In certain implementations, the fragrance dispensing unit can include one or more pushbuttons, wherein the pressing of the button may mechanically or electronically open a vent or release a burst of fragrance. The method simplifies the release process and allows for instant fragrance bursts when needed.

In certain implementations, the fragrance dispensing unit may include a dial mechanism. A small dial can be rotated to disperse the fragrance. The rotation of the dial adjusts the amount of exposed holes area for diffusing the fragrance. The dial can provide more precise control over the fragrance intensity and requires less effort than twisting the entire fragrance unit.

In certain implementations, the fragrance dispensing unit may include a pivot or tilt mechanism for diffusing the fragrance. A pivot or tilt mechanism can be incorporated into the receptacle, wherein the fragrance unit or a plug built into the receptacle can be tilted or opened to release the fragrance.

In certain implementations, the fragrance dispensing unit may include a removable cover or lid, wherein removing the cover exposes the holes, and the fragrance can be released. The cover can be applied again to seal the holes.

In certain implementations, the fragrance dispensing unit can include an Accordion or Bellows mechanism. The fragrance compartment can be designed like an accordion. Compressing or expanding this section adjusts the amount of exposed vent area, said mechanism provides a visually appealing method of adjusting fragrance intensity and ensures a gradual release.

In certain implementations, the fragrance dispensing unit may include a Switch Mechanism. A toggle switch or rocker switch is used to open or close the vents. The switch can have multiple positions to control the release amount. Said mechanism Offers a straightforward approach, with clear “on” and “off” positions. The multi-position switches can offer varying fragrance intensity levels.

In certain implementations, the mechanism can include a Pulley or Cord Mechanism. By pulling a cord or string, the user can adjust a flap or cover over the vents. The length to which the cord is pulled determines the fragrance intensity. This mechanism provides a unique interaction and can allow for very granular control over the vent exposure.

In certain implementations, the container and/or receptacle may be primarily crafted from high-quality, non-reactive plastic, ensuring longevity and resistance to any potential chemical interaction with its contents. The design of the apparatus may not just be functional but also ergonomic. The wall of the fragrance dispensing unit may include some texture or ribs for a better grip, ensuring ease of use even with wet hands. Similarly, the pump mechanism may be designed to require minimal force and reduce strain during use.

In certain implementations, the apparatus may also include a Visual Indicator. A color-changing indicator can be integrated into the twist base. This indicator, possibly made from thermochromic materials, changes color based on the intensity of the fragrance set by the user, offering a quick visual cue.

In certain implementations, the apparatus may also include a lock mechanism for the pump mechanism and fragrance dispensing unit. This may be useful for households with children, wherein a safety lock mechanism can be integrated. When activated, this lock prevents the dispensing of soap and the release of fragrance, ensuring child safety.

In certain implementations, the base of the apparatus can have a non-slip base that may prevent the apparatus from slipping on wet surfaces. For example, the base can be equipped with a non-slip rubber or silicone base.

In certain implementations, the apparatus may include a spray mechanism. Instead of a passive air freshener, a button can be provided which can be manually actuated to disperse a burst of fragrance. Instead of a manual button, a battery-operated mechanism can also be used that periodically releases the fragrance. For example, the fragrance release mechanism can be automatically activated for dispersing a burst of fragrance when the soap is dispensed from the liquid dispensing unit. For example, when the pump mechanism is actuated.

In certain implementations, the disclosed receptacle may include a carousel-style design that has multiple compartments, and a different fragrance can be contained in different compartments. The users can rotate between different fragrances based on preference.

The disclosed apparatus can be used in restrooms. As one of the primary locations for handwashing, incorporating a pleasant aroma ensures not only cleanliness but also a refreshing experience. The combined dispenser can eliminate the need for a separate air freshener unit, decluttering the space.

The disclosed apparatus can also be used in Kitchens. After handling foods like garlic, onions, or fish, washing hands can leave behind lingering scents. The air freshener component actively combats such odors, ensuring the kitchen smells clean and inviting. In many cases, the automatic release of fragrance substance upon actuation of the pump action for dispensing soap can be advantageous. This may fill the atmosphere around the user with aroma and wastage by preventing the release of fragrance in the absence of people.

In certain implementations, the apparatus can also be used in Laundry Rooms. Frequently, laundry rooms are compact spaces where freshness is appreciated. While users are pre-treating clothes or washing hands after handling soiled garments, the dispenser provides both cleanliness and a refreshing atmosphere.

Similar to households, the Restrooms in offices can benefit from a combined dispenser, ensuring employees and visitors experience a pleasant and hygienic environment. Also, in dining establishments, such as café and restaurants, ensuring a pleasant restroom experience reflects on the overall brand. Combining hand soap with an adjustable fragrance ensures visitors have a consistent experience. Guest rooms, suites, and public restrooms can incorporate the disclosed apparatus to enhance the guest experience by providing luxury and practicality in one unit. Public restrooms in malls often need to cater to high footfall. A disclosed apparatus not only ensures hand hygiene but also continuously refreshes the space, enhancing the overall visitor experience. In areas where hand hygiene is paramount, the disclosed apparatus provides the necessary soap and introduces a calming or neutralizing fragrance to clinical settings, making them less intimidating. Dental clinics, known for their distinct clinical smell, can benefit from the air freshener's neutralizing abilities post-handwashing, offering a more pleasant atmosphere for patients.

The disclosed apparatus can also be used in educational institutions, such as schools and colleges, especially those with boarding facilities. The disclosed apparatus can ensure students practice good hygiene while also maintaining a fresh environment in shared facilities. In daycare centers, young children often need reminders to wash their hands. An apparatus that offers soap and emits a pleasant fragrance can make the process more engaging and ensure clean, fresh-smelling environments for both children and caregivers.

The disclosed apparatus can also be used in Spas & Wellness Centers. The very essence of these places is relaxation. After treatments or therapies, clients can wash their hands and simultaneously enjoy a calming fragrance, amplifying the relaxation experience. In Gyms & Fitness Centers, after a workout, members can refresh by washing their hands and simultaneously experience a rejuvenating scent, enhancing the post workout freshness.

The disclosed apparatus saves counter space as now only one unit is needed for both the soap and fragrance. The disclosed apparatus can change the hand wash experience, by ensuring a pleasant aroma accompanies handwashing. Users can adjust fragrance intensity based on their preference.

In certain implementations, the disclosed apparatus may allow the regular release of a small amount of fragrance using any of the methods described above. For example, a few holes in the fragrance dispensing unit can be exposed for the continuous release of a very small amount of fragrance. This may be enough to keep the space feeling fresh. Now, when the apparatus is used for soap dispensing, a burst of fragrance can be released. Thus, the apparatus may include two or more fragrance-release mechanisms. The burst of fragrance can be automatically released upon actuation of the pump for dispensing the soap.

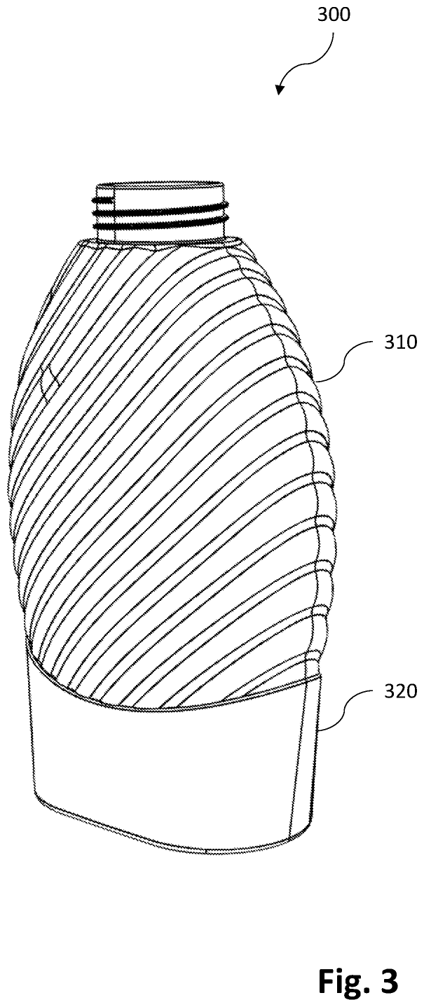

Referring to FIGS. 3 which shows a perspective view of another embodiment of the apparatus 300. The apparatus includes a main container 310 and a fragrance dispensing unit 320. The main container can hold any substance, such as solids or liquids. For example, the container can be used as a soap dispenser. The mount of the container can be closed by a cap. Also, a hand pump can be includes for dispensing the liquid. Thus, the shape, size, and utility of the main container can be varied without departing from the scope of the present invention.

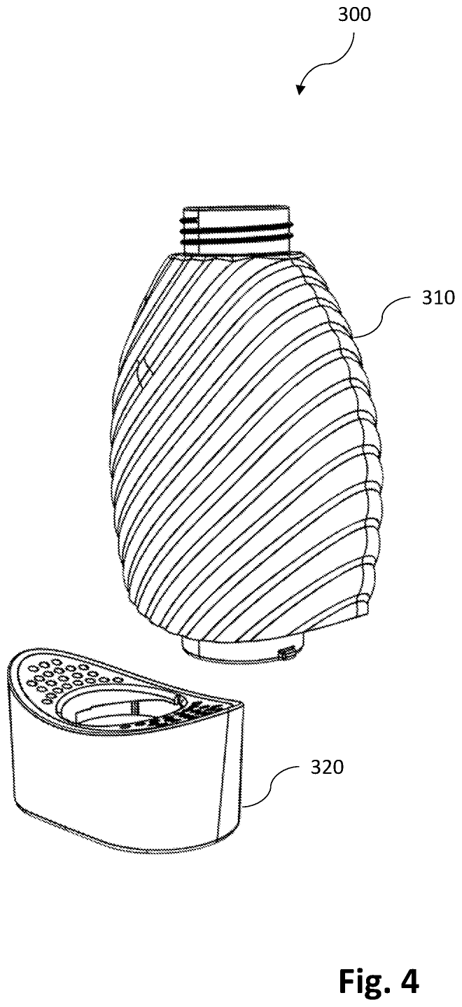

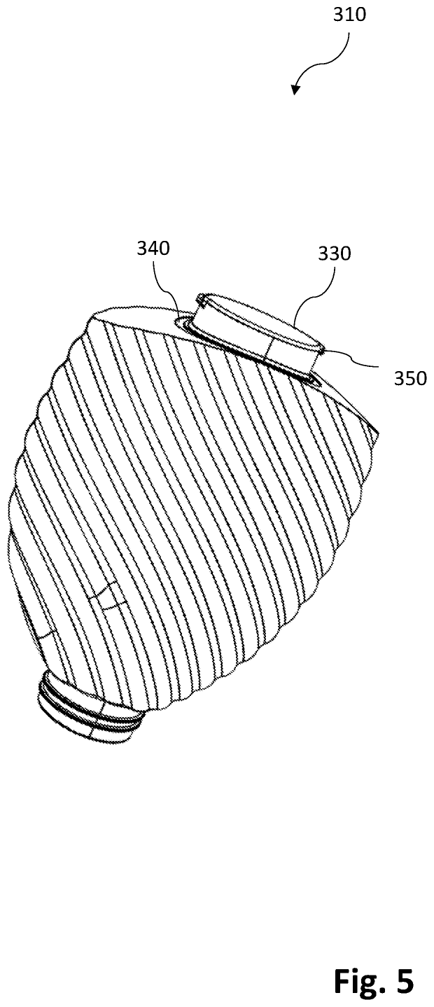

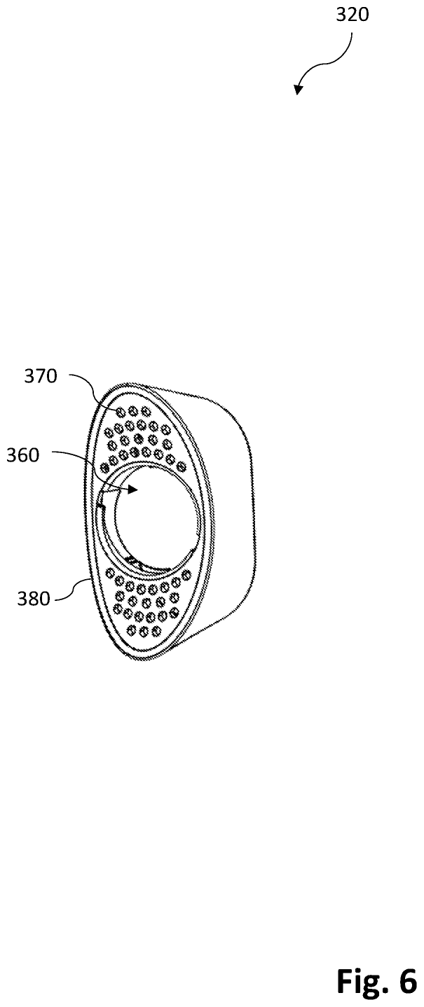

FIG. 4 shows an exploded view of the apparatus 300, FIG. 5 shows a bottom perspective view of the main container, and FIG. 6 shows a top perspective view of the fragrance dispensing unit. The main container on its bottom side includes a bracket 330 that is of a cylindrical shape. The bracket has a base and a skirt along a periphery of the base, wherein the skirt integrates with a base of the container. The base along the skirt has a continuous circular groove 340. The circular grove can receive a rubber ring, O-ring, or gasket, or a similar sealing member to prevent any fragrance leak from the fragrance dispensing unit. The continuous circular groove 340 can be a receiving cavity for a silicone or similar buffer material that helps create an enclosed seal so the fragrance does not leak while closed to maintain the fragrance or when the customer intends to keep it closed when not in use.

The bracket also has male mating members 350 for snap fitting to the liquid dispensing unit. The cylindrical shape of the bracket allows for rotating the fragrance dispensing unit; however, any other shape of the bracket is within the scope of the present invention. Also, the mating member is shown as a snap fitting mechanism, however, any other fastening mechanism is within the scope of the present invention.

The fragrance dispensing unit 320 may include a cavity 360 in which the fragrance substance is contained. The peripheral cavity for the fragrance dispensing unit includes female mating member of the snap fit mechanism. A top wall of the fragrance dispensing unit having the aperture of the cavity can include holes 370 for allowing the fragrance to be dispersed into the environment. A silicon ring along the top wall can provide a seal against the leakage of the fragrance. For releasing the fragrance, the fragrance dispensing unit can be turned to expose the holes.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

What is claimed is:

1. An apparatus comprising:
a liquid dispensing unit, the liquid dispensing unit comprises:
a container; and
a pump for dispensing the liquid contained in the container; and
a fragrance dispensing unit coupled to a bottom of the liquid dispensing unit, wherein the fragrance dispensing unit comprises:
a receptacle configured to contain fragrance substances, the receptacle having a top wall; and
one or more holes in the top wall of the receptacle or the receptacle, wherein the one or more holes are for releasing the fragrance.

2. The apparatus of claim 1, wherein the liquid is soap.

3. The apparatus of claim 1, wherein the one or more holes comprise multiple holes in the top wall of the fragrance dispensing unit.

4. The apparatus of claim 3, wherein the fragrance dispensing unit is configured to rotate from a closed state to up to 90 degrees, wherein the top wall of the fragrance dispensing unit is covered by a bottom of the container in the closed state.

5. The apparatus of claim 4, wherein a degree of rotation of the fragrance dispensing unit relative to the liquid dispensing unit from the closed state up to 90 degrees is proportional to number of holes exposed for releasing the fragrance, wherein the fragrance release increases with rotation of the fragrance dispensing unit.

6. The apparatus of claim 1, wherein each of the container and the receptacle has one or more transparent or translucent windows to check a level of the liquid inside the container and a level of the fragrance substance inside the receptacle.

7. The apparatus of claim 5, wherein the multiple holes in the top wall are sealed in the closed state by the bottom of the container preventing any leakage of the fragrance substance, wherein the liquid dispensing unit comprises a silicone ring along a periphery of the top wall.

8. A method for dispensing a liquid and fragrance, the method comprising:
providing an apparatus comprising:
a liquid dispensing unit, the liquid dispensing unit comprises:
a container; and
a pump for dispensing the liquid contained in the container; and
a fragrance dispensing unit coupled to a bottom of the liquid dispensing unit, wherein the fragrance dispensing unit comprises:
a receptacle configured to contain fragrance substance;
a lid covering an opening in the receptacle; and
one or more holes in a wall of the receptacle or the lid, wherein the one or more holes are for release of the fragrance;
dispensing the liquid from the liquid dispensing unit; and
dispensing, continuously or periodically, fragrance by diffusion in air, from the fragrance dispensing unit.

9. The method of claim 8, wherein the liquid is a soap.

10. The method of claim 8, wherein the one or more holes comprise multiple holes in the lid of the fragrance dispensing unit.

11. The method of claim 10, wherein the fragrance dispensing unit is configured to be rotated from a closed state to up to 90 degrees, wherein the lid of the fragrance dispensing unit is covered by a bottom of the container in the closed state, wherein the method further comprises:
rotating, the fragrance dispensing unit, from the closed state to expose the holes.

12. The method of claim 11, wherein a degree of rotation of the fragrance dispensing unit relative to the liquid dispensing unit from the closed state up to 90 degrees is proportion to a number of holes exposed for releasing the fragrance, wherein fragrance release increases with rotation of the fragrance dispensing unit.
